(19) United States
(12) Patent Application Publication
(10) Pub. No.: US 2023/0355553 A1
(43) Pub. Date: Nov. 9, 2023

(54) COMPOSITIONS AND METHODS FOR TREATING AND/OR PREVENTING OCULAR DISORDERS

(71) Applicant: University of Virginia Patent Foundation, Charlottesville, VA (US)

(72) Inventors: Jayakrishna Ambati, Charlottesville, VA (US); Ivana Apicella, Charlottesville, VA (US); Meenakshi Ambati, Charlottesville, VA (US); Siddharth Narendran, Charlottesville, VA (US); Bradley David Unti Gelfand, Charlottesville, VA (US); Shao-bin Wang, Charlottesville, VA (US); Felipe Pereira, Charlottesville, VA (US)

(73) Assignee: University of Virginia Patent Foundation, Charlottesville, VA (US)

(21) Appl. No.: 17/799,149

(22) PCT Filed: Feb. 11, 2021
(86) PCT No.: PCT/US2021/017663

§ 371 Date: Aug. 11, 2022

Related U.S. Application Data
(60) Provisional application No. 62/972,848, filed on Feb. 11, 2020.

(51) Int. Cl. A61K 31/138; A61P 27/12

(52) U.S. Cl. CPC A61K 31/138; A61P 27/12

(57) ABSTRACT

Provided are methods for treating macular degeneration, optionally macular degeneration, in subject in need thereof. In some embodiments, the methods include administering to a subject in need thereof a therapeutically effective amount of fluoxetine in an amount and via a route sufficient to treat or prevent development of macular degeneration in the subjects. Also provided are methods for inhibiting activation of an NLRP3-ASC inflammasome in the subjects, inhibiting ASC speck formation in the subjects, inhibiting Alu RNA-induced RPE degeneration in the subjects, and treatment and/or prevention of the development of diseases, disorders, and/ or conditions.

### CROSS REFERENCE TO RELATED APPLICATION

The presently disclosed subject matter claims the benefit of U.S. Provisional blood glucose measuring system Application Serial No. 62/972,848, filed Feb. 11, 2020, the disclosure of which incorporated herein by reference in its entirety.

### GOVERNMENT INTEREST

This invention was made with government support under Grant Nos. EY028027 and EY031039 awarded by The National Institutes of Health. The government has certain rights in the invention.

### BACKGROUND

Age related macular degeneration (AMD) is a progressive eye condition affecting millions of people worldwide. In particular, it is the number one cause of vision loss and legal blindness in adults over 60 in the United States. As the population continues to age, the incidence of AMD is also expected to rise. The disease affects the macula of the eye, where the sharpest central vision occurs. Although it rarely results in complete blindness, it robs the individual of all but the outermost, peripheral vision, leaving only dim images or black holes at the center of vision.

Macular degeneration is categorized as either dry (i.e., atrophic) or wet (i.e., neovascular). The dry form occurs in about 90% of AMD patients, although the wet form of AMD typically results in more serious vision loss.

Dry macular degeneration affects nearly 200 million people worldwide (Wong et al., 2014). There is no FDA-approved therapy for this disease, which is the leading cause of irreversible blindness among people over 50 years of age (Mitchell et al., 2018). Vision loss in this condition results from degeneration of the retinal pigmented epithelium (RPE). RPE cell death in dry macular degeneration is driven by accumulation of toxic molecules known as Alu RNAs, which are noncoding RNAs in the human genome (Kaneko et al., 2011). Alu RNA induces RPE degeneration by activating a macromolecular protein complex known as the NLRP3-ASC inflammasome (Tarallo et al., 2012).

Typical treatments for wet macular degeneration include photodynamic therapy (PDT) and anti-VEGF injection. In PDT, a light-activated drug such as verteporfin is injected intravascularly and when the light-activated material reaches newly formed blood vessels of the retina a laser is used to activate the drug in the eyes to selectively destroy the newly formed blood vessels. However, recurrence is common even after this treatment, so repeated treatments are typically required. A frequent side effect of the treatments is damage to the retina, however.

Another treatment for wet macular degeneration involves the use of anti-vascular endothelial growth factor (VEGF) antibodies. Generally, an intravitreal injection of an anti-VEGF antibody is employed in an attempt to inhibit the formation and proliferation of new blood vessels by inhibiting selectively binding to and thus inhibiting VEGF signaling. One such antibody is Avastin, which is approved for the treatment of cancer and is used for eye disease. However, anti-VEGF injections are costly and inconvenient because it requires direct injection through the eye. It also needs a regular treatment once a month, which can result in a risk of bleeding, pain, infection, retinal detachment, etc.

Therefore, newer approaches to treating macular degeneration have been investigated. Such approaches include employing a neuroprotectant that inhibits the death of the retinal cells causing macular degeneration, and use of inhibitors of receptor-interacting serine/threonine-protein kinase 1 activity (RIPK1) such as Necrostatin-1 (Nec-1; R)-5-([7-chloro-1H-indol-3-yl]methyl)-3-methylimidazolidine-2,4-dione; see e.g., Trichonas et al., 2010; Murakami et al., 2011; Dong et al., 2012; Sato et al., 2013). Additional RIPK1 inhibitors are disclosed in U.S. Pat. No. 10,501,473, which is incorporated herein by reference in its entirety. These approaches also suffer from various shortcomings, and thus new approaches to treat and/or prevent macular degeneration are still needed.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter relates in some embodiments to methods for treating and/or preventing development of macular degeneration in subjects in need thereof. In some embodiments, the methods comprise, consist essentially of, or consist of administering to a subject in need thereof one or more doses of fluoxetine in an amount and via a route sufficient to treat or prevent development of macular degeneration in the subject. In some embodiments, the macular degeneration is dry macular degeneration.

The presently disclosed subject matter also relates in some embodiments to methods for inhibiting activation of NLRP3-ASC inflammasomes in subjects. In some embodiments, the methods comprise, consist essentially of, or consist of administering to a subject one or more doses of fluoxetine in an amount and via a route sufficient to inhibit activation of an NLRP3-ASC inflammasome in the subject. In some embodiments, the NLRP3-ASC inflammasome is present in retinal pigmented epithelium (RPE) cells and/or macrophages in the subject.

The presently disclosed subject matter also relates in some embodiments to methods for inhibiting ASC speck formation in subjects. In some embodiments, the methods comprise, consist essentially of, or consist of administering to a subject one or more doses of fluoxetine in an amount and via a route sufficient to inhibit ASC speck formation in the subject.

The presently disclosed subject matter also relates in some embodiments to methods for inhibiting Alu RNA-induced RPE degeneration in subjects. In some embodiments, the methods comprise, consist essentially of, or consist of administering to a subject one or more doses of fluoxetine in an amount and via a route sufficient to inhibit Alu RNA-induced RPE degeneration in the subject.

The presently disclosed subject matter also relates in some embodiments to methods for treating and/or preventing development of diseases, disorders, and/or conditions associated with NLRP3-ASC inflammasome activation in subjects in need thereof. In some embodiments, the methods comprise, consist essentially of, or consist of administering to a subject one or more doses of fluoxetine in an amount and via a route sufficient to treat or prevent development of a disease, disorder, and/or condition in the subject. In some embodiments, the disease, disorder, and/or condition associated with NLRP3-ASC inflammasome activation is selected from the group consisting of arthritis, optionally rheumatoid arthritis and/or reactive arthritis, diabetes mellitus, chronic obstructive pulmonary disease, inflammatory bowel disease, irritable bowel syndrome, Duchenne muscular dystrophy, graft-versus-host disease, chronic pain, proliferative vitreoretinopathy, glaucoma, multiple sclerosis, bipolar disorder, major depressive disorder, renal fibrosis, nephritis, pulmonary fibrosis, Huntington’s disease, osteoporosis, chronic lymphocytic leukemia, anxiety disorders, pulmonary tuberculosis, osteoporosis, optionally osteoporosis in post-menopausal women and/or fracture patients, systemic lupus erythematosus, discoid lupus erythematosus, chronic inflammatory and neuropathic pain, autosomal dominant polycystic kidney disease, spinal cord injury, Alzheimer’s disease, neuropathic pain, hypertension, varicose veins, type I diabetes, type II diabetes, gout, autoimmune hepatitis, graft vascular injury, atherosclerosis, thrombosis, metabolic syndrome, salivary gland inflammation, traumatic brain injury, ischemic heart disease, ischemic stroke, Parkinson’s disease, melanoma, neuroblastoma, prostate, breast, skin, and thyroid cancers, tubular early gastric cancer, neuroendocrine cancer, mucoid colon cancer, colon cancer; high-grade urothelial carcinoma, kidney clear cell carcinoma, undifferentiated ovary carcinoma, papillary intracystic breast carcinoma, gram negative sepsis, infectious Pseudomonas aeruginosa, Vibrio cholera, Legionella spp., Francisella spp., and Leishmania spp. Chlamydia spp., cryopyrinopathies; keratitis, acne vulgaris, Crohn’s disease, ulcerative colitis, insulin resistance, obesity, hemolytic-uremic syndrome, polyoma virus infection, immune complex renal disease, acute tubular injury, lupus nephritis, familial cold autoinflammatory syndrome, Muckle-Wells syndrome and neonatal onset multisystem inflammatory disease, chronic infantile neurologic cutaneous and articular autoinflammatory diseases, renal ischemia-perfusion injury, glomerulonephritis, cryoglobulinemia, systemic vasculitides, IgA nephropathy, malaria, helminth parasites, septic shock, allergic asthma, hay fever, drug-induced lung inflammation, contact dermatitis, leprosy, Burkholderia cenocepacia infection, respiratory syncitial virus infection, psoriasis, scleroderma, cystic fibrosis, syphilis, Sjögren’s syndrome, inflammatory joint disease, non-alcoholic fatty liver disease, sterile liver inflammation, cardiac surgery (peri-/post-operative inflammation), acute and chronic organ transplant rejection, acute and chronic bone marrow transplant rejection, tumor angiogenesis, amyotrophic lateral sclerosis, and autism spectrum disorder.

The presently disclosed subject matter also relates in some embodiments to methods for preventing and/or treating RPE degeneration in subjects in need thereof. In some embodiments, the methods comprise, consist essentially of, or consist of introducing into a subject’s eye(s), retina(s), and/or RPE cells present therein an effective amount of a composition comprising fluoxetine, optionally wherein the composition comprises fluoxetine in a substantially enantiomerically pure form. In some embodiments, the RPE degeneration is associated with age-related macular degeneration (AMD), optionally dry AMD.

In some embodiments of the presently disclosed methods, the fluoxetine is substantially enantiomerically pure R-fluoxetine or substantially enantiomerically pure S-fluoxetine.

In some embodiments, the presently disclosed subject matter relates to methods for reducing the incidence of cataract formation in subjects receiving fluoxetine. In some embodiments, the methods comprise, consist essentially of, or consist of administering the fluoxetine in a substantially pure enantiomeric form rather than as a racemic mixture.

The presently disclosed subject matter also relates in some embodiments to uses of compositions comprising, consisting essentially of, or consisting of fluoxetine to treat and/or prevent development of diseases, disorders, and/or conditions in subjects in need thereof. In some embodiments, the disease, disorder, and/or condition is a disease, disorder, and/or condition of the eye. In some embodiments, the disease, disorder, and/or condition is a disease, disorder, and/or condition of the retina. In some embodiments, the disease, disorder, and/or condition is a disease, disorder, and/or condition of the RPE. In some embodiments, wherein the disease, disorder, and/or condition is associated with RPE degeneration.

The presently disclosed subject matter also relates in some embodiments to uses of compositions comprising, consisting essentially of, or consisting of fluoxetine to treat and/or prevent development of macular degeneration in subjects in need thereof.

The presently disclosed subject matter also relates in some embodiments to uses of compositions comprising, consisting essentially of, or consisting of fluoxetine to treat or prevent development of a disease, disorder, and/or condition in a subject in need thereof, wherein the disease, disorder, and/or condition is associated with NLRP3-ASC inflammasome activation.

In some embodiments of the presently disclosed uses, the fluoxetine is substantially enantiomerically pure R-fluoxetine or substantially enantiomerically pure S-fluoxetine.

The presently disclosed subject matter also relates in some embodiments to uses of compositions comprising, consisting essentially of, or consisting of the subject is a human.

The presently disclosed subject matter also relates in some embodiments to pharmaceutical compositions for preventing and/or treating diseases, disorders, and/or conditions associated with RPE degeneration and/or NLRP3-ASC inflammasome activation in subjects. In some embodiments, the pharmaceutical compositions comprise, consist essentially of, or consists of an effective amount of fluoxetine. In some embodiments, the fluoxetine is substantially enantiomerically pure R-fluoxetine or substantially enantiomerically pure S-fluoxetine. In some embodiments, the disease, disorder, and/or condition associated with RPE degeneration and/or NLRP3-ASC inflammasome activation is selected from the group consisting of DICER1 syndrome, type 2 diabetes mellitus, diabetic retinopathy, age-related macular degeneration (AMD), aberrant choroidal and retinal neovascularization (CRNV), subretinal and retinal fibrosis, Fuchs’ endothelial corneal dystrophy, Alzheimer’s disease, rheumatoid arthritis, lupus, renal injury, tubulointerstitial fibrosis, glial axonal degeneration, idiopathic pulmonary fibrosis, lipid dysregulation, cholesterol accumulation associated with non-alcoholic steatohepatitis, clear cell renal cell carcinoma, atopic dermatitis, glomerulopathy, disorders of hypomyelination, tubal ectopic pregnancy and tubal abnormalities such as but not limited to cysts and disorganization of epithelial cells and smooth muscle cells, amyotrophic lateral sclerosis (ALS), Duchenne’s muscular dystrophy, Sertoli cell deficiency/impaired spermatogenesis, and combinations thereof. In some embodiments, the disease, disorder, and/or condition is age-related macular degeneration (AMD). In some embodiments, the pharmaceutical composition is formulated for administration by intravitreous injection; subretinal injection; episcleral injection; sub-tenon injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; and any combination thereof.

Accordingly, it is an object of the presently disclosed subject matter to provide compositions and methods for preventing and/or treating diseases, disorders, and/or conditions associated with RPE degeneration and/or NLRP3-ASC inflammasome activation of the eye, including but not limited to AMD.

These and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and EXAMPLES.

### BRIEF DESCRIPTIONS OF THE FIGURES

FIG. 1 is a representative western blot image showing Alu RNA (SEQ ID NO: 1) induced cleavage of pro-caspase-1 (50 kDa) into active caspase-1 (20 kDa) in mouse bone marrow derived macrophages, and that Fluoxetine (FLX) reduced this caspase-1 activation. N = 3 per group.

FIG. 2 is a representative western blot image showing Alu RNA (SEQ ID NO: 1) induced cleavage of pro-caspase-1 (50 kDa) into active caspase-1 (20 kDa) in human ARPE19 cells, and that Fluoxetine (FLX) reduced this caspase-1 activation. N = 3 per group.

FIGS. 3A and 3B are bar graphs summarizing the results of ELISA analysis of secreted IL-1β from mouse bone marrow derived macrophages. Alu RNA (SEQ ID NO: 1; FIG. 3A) or B2 RNA (SEQ ID NO: 2; FIG. 3B) induced secretion of this cytokine, and Fluoxetine (FLX) reduced its secretion. N = 3 per group. P value from two-tailed Student’s t test. ND, not detected. -: absent. +: present.

FIGS. 4A and 4B are bar graphs summarizing the results of ELISA analysis of secreted IL-18 from human ARPE-19 cells. Alu RNA (SEQ ID NO: 1; FIG. 4A) or B2 RNA (SEQ ID NO: 2; FIG. 4B) induced secretion of this cytokine, and Fluoxetine (FLX) reduced its secretion. N = 3 per group. P value from two-tailed Student’s t test. ND, not detected. -: absent. +: present.

FIGS. 5A and 5B summarize the results of experiments testing the ability of fluoxetine (FLX) to inhibit inflammasome activation. FIG. 5A are representative immunofluorescent images of apoptosis-associated speck-like protein containing a CARD (ASC) specks (examples are stained red in a color photograph and are circled in white for ease of identification) in bone marrow derived macrophages (BMDMs) treated with Alu RNA (SEQ ID NO: 1) or Alu RNA (SEQ ID NO: 1) plus FLX. FIG. 5B is a bar graph of mean and SEM. N = 5 per group for the number of specks per 40X field. *P = 0.006 (Alu RNA + FLX vs. Alu RNA), Student’s two-tailed t test.

FIGS. 6A and 6B show the results of experiments testing the abilities of various antidepressant drugs to inhibit RPE degeneration. FIG. 6A are representative ZO-1-stained (appears red in color photographs) RPE flat mounts of mice 7 days after PBS, Alu RNA (SEQ ID NO: 1) + PBS, or Alu RNA (SEQ ID NO: 1) + FLX injections. Intravitreous injection of FLX or other antidepressants was performed 1 day before and immediately after subretinal Alu RNA (SEQ ID NO: 1) injection. FIG. 6B is a bar graph summarizing the results expressed as the percentage of eyes with RPE degeneration in each group. N = 6-10 eyes per group. *P = 0.007 (Alu RNA + FLX vs. Alu RNA + PBS), Two-tailed z-test for proportions. PBS, phosphate-buffered saline; FLX, fluoxetine; CIT, Citalopram; PAR, Paroxetine; SER, Sertraline; ESC, Escitalopram; VEN, Venfaximine; IMI, Imipramine; AMI, Amitriptyline; AGO, Agomelatine.

FIG. 7 is a Kaplan-Meier Survival curve showing that Fluoxetine use was associated with a slower rate of developing dry macular degeneration. P < 0.0001 by log rank test.

FIG. 8 is a series of fundus photos of the retina of mice injected with Alu RNA (SEQ ID NO: 1) and fluoxetine (racemic), R-fluoxetine enantiomer, or S-Fluoxetine enantiomer. Arrowheads show extent and intensity of degeneration of the retinal pigmented epithelium (RPE). Alu RNA (SEQ ID NO: 1) induced extensive and intense RPE degeneration (white discoloration where the arrowheads are present).

FIG. 9 is a series of fundus photos of the retina of mice injected with oligomerized amyloid beta and fluoxetine. Arrowheads show extent and intensity of degeneration of the retinal pigmented epithelium (RPE). Amyloid beta induced extensive and intense RPE degeneration (white discoloration where the arrowheads are present).

### DETAILED DESCRIPTION

Disclosed herein is the discovery that fluoxetine, an FDA-approved drug for treating clinical depression, inhibits activation of the NLRP3-ASC inflammasome in retinal pigmented epithelium (RPE) cells and macrophages, two critical cell types in dry macular degeneration. Also demonstrated is that fluoxetine, unlike several other antidepressant drugs, inhibits RPE degeneration in an in vivo model of dry macular degeneration. Finally, a Big Data analysis of a health insurance database of 25 million Americans is presented, in which a dramatic reduction in the development of dry macular degeneration among patients with depression who were treated with fluoxetine compared to those treated with other anti-depressants was identified. Collectively, these studies triangulate to link fluoxetine as a potential drug repurposing candidate that could become the first therapy for a major unmet medical need that causes blindness in millions of people in the United States and across the world.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some embodiments all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

With regard to administering a composition, the term “administering” as used herein refers to any method for providing a composition and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-tenon administration, peribulbar administration, transscleral drug delivery, intraocular injection, intravenous injection, intraparenchymal/intracranial injection, intraarticular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

As used herein, “amino acids” are represented by the full name thereof, by the three letter code corresponding thereto, or by the one-letter code corresponding thereto, as indicated in Table 1:

TABLE 1

Amino Acids, Abbreviations Thereof, and Functionally Equivalent Codons

| | | | |
|---|---|---|---|
| Full Name | 3-Letter Code | 1-Letter Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation, and/or substitution with other chemical groups which can change the peptides’ circulating half-lives without adversely affecting their activities. Additionally, a disulfide linkage may be present or absent in the peptides of the presently disclosed subject matter.

The term “amino acid” is used interchangeably with “amino acid residue”, and may refer to a free amino acid and/or to an amino acid residue of a peptide. It will be apparent from the context in which the term is used whether it refers to a free amino acid or a residue of a peptide.

Amino acids have the following general structure:

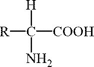

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group. Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-α-hydroxylysyl and D-α-methylalanyl, L-α-methylalanyl, β-amino acids, and isoquinolyl. D-amino acids and/or non-naturally occurring synthetic amino acids can also be incorporated into the peptides of the presently disclosed subject matter. Other derivatives include replacement of the naturally occurring side chains of the 20 genetically encoded amino acids (or any L- or D- amino acid) with other side chains.

As used herein, the term “silent mutation” refers to one or more nucleotide changes that in the context of a coding sequence do not result in an amino acid change in the polypeptide encoded by the coding sequence. One of ordinary skill in the art can determine silent mutations for most although not all of the naturally occurring amino acids by reference to the genetic code summarized in the Table above, particularly the functionally equivalent codons. Silent mutations can involve single nucleotide changes (e.g., GAC to GAU or vice versa for aspartic acid; a change of one of CGA, CGC, CGG, or CGU to one of the other three for arginine; a change of one of ACA, ACC, ACG, or ACU to one of the other three for threonine; etc.). However, silent mutations need not be single nucleotide changes. By way of example and not limitation, the codons ACG, AGU, UCA, UCC, UCG, and UCU all code for serine, so a change from ACG to AGU or even from to AGU to UCG would constitute a silent mutation as that term is used herein.

As used herein, the term “conservative amino acid substitution” is defined herein as exchanges within one of the following five groups:
I. Small aliphatic, nonpolar or slightly polar residues: Ala, Ser, Thr, Pro, Gly;
II. Polar, negatively charged residues and their amides: Asp, Asn, Glu, Gln;
III. Polar, positively charged residues: His, Arg, Lys;
IV. Large, aliphatic, nonpolar residues: Met Leu, Ile, Val, Cys
V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

With respect to the terms “comprising”, “consisting of”, and “consisting essentially of”, where one of these three terms is used herein, the presently disclosed and claimed subject matter can include the use of either of the other two terms. For example, in some embodiments, the presently disclosed subject matter relates to compositions comprising antibodies. It would be understood by one of ordinary skill in the art after review of the instant disclosure that the presently disclosed subject matter thus encompasses compositions that consist essentially of the antibodies of the presently disclosed subject matter, as well as compositions that consist of the antibodies of the presently disclosed subject matter.

As used herein, the terms “condition”, “disease condition”, “disease”, “disease state”, and “disorder” refer to physiological states in which diseased cells or cells of interest can be targeted with the compositions of the presently disclosed subject matter. By way of example and not limitation, a “condition”, “disease condition”, “disease”, “disease state”, and “disorder” of the presently disclosed subject matter relates to a physiological state in which the diseased cell or cells of interest are cells of the eye, optionally RPE cells.

A “disease” is a state of health of an animal wherein the animal cannot maintain homeostasis, and wherein if the disease is not ameliorated then the animal’s health continues to deteriorate.

In contrast, a “disorder” in an animal is a state of health in which the animal is able to maintain homeostasis, but in which the animal’s state of health is less favorable than it would be in the absence of the disorder. Left untreated, a disorder does not necessarily cause a further decrease in the animal’s state of health.

As used herein, the term “diagnosis” refers to detecting a risk or propensity to a condition, disease, or disorder. In any method of diagnosis exist false positives and false negatives. Any one method of diagnosis does not provide 100% accuracy.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein, the term “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids that have been substantially purified from other components which naturally accompany the nucleic acid it in a cell, e.g., RNA or DNA or proteins. The term therefore includes, for example, a recombinant DNA which is incorporated into a vector, an autonomously replicating plasmid or virus, or the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the presently disclosed subject matter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

As used herein, the term “nucleic acid” also encompasses RNA as well as single and double-stranded DNA and cDNA. Furthermore, the terms “nucleic acid”, “DNA”, “RNA”, and similar terms also include nucleic acid analogs, e.g., the so-called “peptide nucleic acids”, which are known in the art and have peptide bonds instead of phosphodiester bonds in the backbone, are considered within the scope of the presently disclosed subject matter.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, which in some embodiments are no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two or more polynucleotides as “operably linked” it is meant that a single-stranded or double-stranded nucleic acid comprises the two or more polynucleotides arranged within a nucleic acid molecule in such a manner that at least one of the two or more polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, subcutaneous, intraperitoneal, intramuscular, and intrasternal injection.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide (e.g., polymerization). Such synthesis occurs when the polynucleotide primer is placed under conditions in which synthesis is induced, e.g., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. In some embodiments, primers can be labeled, e.g., with chromogenic, radioactive, and/or fluorescent moieties and used as detectable moieties.

As used herein, the term “promoter/regulatory sequence” means a nucleic acid sequence which is required for expression of a gene product operably linked to the promoter/regulator sequence. In some embodiments, this sequence may be the core promoter sequence, and in some embodiments, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not generally found joined together in nature. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide produces a “recombinant polypeptide”.

A “recombinant polypeptide” is one which is produced upon expression of a recombinant polynucleotide, in some embodiments by a recombinant host cell.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “polynucleotide” as used herein includes but is not limited to DNA, RNA, complementary DNA (cDNA), messenger RNA (mRNA), ribosomal RNA (rRNA), small hairpin RNA (shRNA), small nuclear RNA (snRNA), short nucleolar RNA (snoRNA), microRNA (miRNA), genomic DNA, synthetic DNA, synthetic RNA, and/or tRNA.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

As used herein, “substantially homologous amino acid sequences” includes those amino acid sequences which have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99%, or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: in some embodiments in 7% sodium dodecyl sulfate SDS, 0.5 M NaPO₄, 1 mM EDTA at 50° C. with washing in 2X standard saline citrate (SSC), 0.1% SDS at 50° C.; in some embodiments in 7% (SDS), 0.5 M NaPO₄, 1 mM EDTA at 50° C. with washing in 1X SSC, 0.1% SDS at 50° C.; in some embodiments in 7% SDS, 0.5 M NaPO₄, 1 mM EDTA at 50° C. with washing in 0.5X SSC, 0.1% SDS at 50° C.; and in some embodiments in 7% SDS, 0.5 M NaPO₄, 1 mM EDTA at 50° C. with washing in 0.1X SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a,b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

As used herein, the term “transduction” refers to the introduction of a foreign nucleic acid into a cell using a vector, in some embodiments a viral vector.

As used herein, the term “transfection” as used herein refers to the introduction of a foreign nucleic acid into a cell using recombinant DNA technology. The term “transformation” means the introduction of a “foreign” (i.e., extrinsic or exogenous) gene, DNA, or RNA sequence to a host cell, such that the host cell will express the introduced gene or sequence to produce a desired substance, such as a protein or enzyme, coded by the introduced gene or sequence. The introduced gene or sequence can also be called a “cloned”, “foreign”, or “heterologous” gene or sequence or a “transgene”, and can include regulatory and/or control sequences, such as start, stop, promoter, signal, secretion, or other sequences used by a cell’s genetic machinery. The gene or sequence can include nonfunctional sequences or sequences with no known function. A host cell that receives and expresses introduced DNA or RNA has been “transformed” and is a “transformant” or a “clone”, and is “transgenic”. The DNA or RNA introduced to a host cell can come from any source, including cells of the same genus or species as the host cell, or cells of a different genus or species

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

As used herein, the terms “operatively linked” and “operably linked refer to transcriptional regulatory elements (such as, but not limited to promoter sequences, transcription terminator sequences, etc.) that are connected to a nucleotide sequence (for example, a coding sequence or open reading frame) in such a way that the transcription of the nucleotide sequence is controlled and regulated by that transcriptional regulatory element. Similarly, a nucleotide sequence is said to be under the “transcriptional control” of a promoter to which it is operably linked. Techniques for operatively linking a promoter region to a nucleotide sequence are known in the art.

### II. Compositions

### II.A. Generally

In some embodiments, the presently disclosed subject matter relates to compositions that comprise, consist essentially of, or consists of inhibitors of macular degeneration. As used herein the term “inhibit” refers to the ability of a compound or any agent to reduce and/or impede a described function and/or pathway. For example, inhibition can be by at least 10%, by at least 25%, by at least 50%, by at least 75%, by at least 80%, by at least 85%, by at least 90%, by at least 95%, by at least 97%, by at least 99%, or more.

A compound or agent that is capable of reducing and/or impeding a described function and/or pathway is thus an “inhibitor” of that function and/or pathway. Similarly, if the described function and/or pathway is associated with a predisposition to and/or development and/or worsening of any disease, disorder, and/or condition and/or any symptom and/or consequence thereof, the inhibitor can also function as an inhibitor of the disease, disorder, and/or condition and/or the symptom and/or consequence thereof. In the context of a disease, disorder, and/or condition and/or the symptom and/or consequence thereof, it is understood that the inhibition can be temporary or incomplete. In such embodiments, the inhibitor might not completely prevent the initiation, development, and/or progression of the disease, disorder, and/or condition and/or the symptom and/or consequence thereof but might delay its onset and/or slow the rate at which it develops and/or progresses. Thus, just as the degree to which the inhibitor might reduce and/or impede a biological function and/or pathway can be less than 100%, the degree to which the inhibitor delays or prevents the initiation, development, and/or progression of the disease, disorder, and/or condition and/or the symptom and/or consequence thereof and/or slows a rate at which it develops and/or progresses can be less than 100%. In some embodiments, a compound or agent is considered an inhibitor if its use results in any measurable improvement in the initiation, development, and/or progression of a disease, disorder, and/or condition and/or at least one symptom and/or consequence thereof, including but not limited to reducing a rate at which such a one symptom and/or consequence of the disease, disorder, and/or condition develops and/or progresses,

In some embodiments, an inhibitor of the presently disclosed subject matter is an inhibitor of macular degeneration and/or a symptom and/or consequence thereof. In some embodiments, the inhibitor of macular degeneration and/or a symptom and/or consequence thereof comprises, consists essentially of, or consists of fluoxetine. Fluoxetine is described in U.S. Pat. Nos. 4,018,895 and 4,194,009, each of which is incorporated herein by reference in its entirety.

In some embodiments, an inhibitor of macular degeneration and/or a symptom and/or consequence thereof can be employed by administration to a subject in need thereof.

### II.B. Pharmaceutical Compositions

In some embodiments, the compositions of the presently disclosed subject matter are provided as part of a pharmaceutical composition. As used herein, the term “pharmaceutical composition” refers to a composition comprising at least one active ingredient (e.g., an inhibitor of the presently disclosed subject matter), whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

In some embodiments, a pharmaceutical composition of the presently disclosed subject matter comprises, consists essentially of, or consists of at least one active ingredient (e.g., an inhibitor of the presently disclosed subject matter) and a pharmaceutically acceptable diluent and/or excipient. As used herein, the term “pharmaceutically acceptable” refers to physiologically tolerable, for either human or veterinary application. Similarly, “pharmaceutical compositions” include formulations for human and veterinary use. The term “pharmaceutically acceptable carrier” also refers to a chemical composition with which an appropriate compound or derivative can be combined and which, following the combination, can be used to administer the appropriate compound to a subject. In some embodiments, a pharmaceutically acceptable diluent and/or excipient is pharmaceutically acceptable for use in a human.

In some embodiments, the pharmaceutical compositions of the presently disclosed subject matter are for use in preventing and/or treating a disease or disorder associated with abnormal physiology in the eye, optionally the retina, further optionally the RPE, of a subject in need thereof. In some embodiments, the disease or disorder of the eye is associated with RPE degeneration, aberrant choroidal and retinal neovascularization (CRNV), or both. In some embodiments, the effective amount inhibits degradation of cells in the eye, optionally the retina, further optionally RPE, of the subject.

The pharmaceutical compositions of the presently disclosed subject matter can in some embodiments consist of the active ingredient alone, in a form suitable for administration to a subject, or the pharmaceutical composition can in some embodiments comprise or consist essentially of the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt refers to an ester or salt form of the active ingredient which is compatible with any other ingredients of the pharmaceutical composition, which is not deleterious to the subject to which the composition is to be administered.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multidose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

As used herein, the term “fluoxetine” refers to N-methyl-3-phenyl-3-[4-(trifluoromethyl)phenoxy]propan-1-amine (CAS No. 54910-89-3). It is generally employed as an antidepressant of the selective serotonin reuptake inhibitor (SSRI) class. The preparation that is generally employed is an equimolar racemic mixture of R-fluoxetine and S-fluoxetine (Gram, 1994). However, it has been determined that the individual enantiomers retain the activities of the racemic mixture but in some embodiments are characterized by fewer side effects. For example, it has been determined that use of the racemic mixture can cause cataracts in up to 50% of treated subjects, whereas use of either enantiomer in pure or substantially pure form reduces or eliminates cataract formation.

Therefore, in some embodiments the fluoxetine is provided as a pure or substantially pure enantiomeric form. As used herein, the phrase “substanitally pure enantiomeric form” refers to a compound that is substantially free from other enantiomers or stereoisomers of the compound (i.e., in enantiomeric excess). In other words, an “S” form of the compound is substantially free from the “R” form of the compound and is, thus, in enantiomeric excess of the “R” form. The terms “substantially pure enantiomeric form”, “pure enantiomer”, and grammatical variants thereof denotes that one enantiomeric form or the other is present in a composition in an amount of more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the composition. In some embodiments, the weights are based upon total weight of all enantiomers or stereoisomers of the compound in a given composition.

### II.B.1. Formulations

The compositions of the presently disclosed subject matter thus comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

Controlled- or sustained-release formulations of a pharmaceutical composition of the presently disclosed subject matter can be made using conventional technology. A formulation of a pharmaceutical composition of the invention suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl parahydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion.

The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for parenteral administration, including but not limited to intraocular injection.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane dial, for example.

Other acceptable diluents and solvents include, but are not limited to, Ringer’s solution, isotonic sodium chloride solution, and fixed oils such as synthetic mono or diglycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt. Formulations suitable for nasal administration may, for example, comprise from about as little as 0.1 % (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, can in some embodiments have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985) Remington’s Pharmaceutical Sciences, Mack Publishing Co., Easton, Pennsylvania, United States of America, which is incorporated herein by reference in itsz entirety.

### II.B.2. Administration

With regard to administering a composition of the presently disclosed subject matter, methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-tenon administration, peribulbar administration, transscleral drug delivery, intraocular injection, intravenous injection, intraparenchymal/intracranial injection, intra-articular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

Suitable methods for administration of the compositions of the presently disclosed subject matter include, but are not limited to intravitreous injection; subretinal injection; episcleral injection; sub-tenon injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; and any combination thereof. Exemplary methods for administering compositions to the eye include those described in, for example, U.S. Pat. Nos. 7,745,389; 8,664,176; 9,314,453; 10,004,788; and 10,117,931, each of which is incorporated herein by reference in its entirety. By way of example and not limitation, methods for intraocular administration of compounds are known and include, but are not limited to injection including but not limited to intravitreal injection (see e.g., U.S. Pat. Nos. 7,678,078; 8,642,067; and 9,144,516; see also U.S. Pat. Application Publication No. 2006/0141049). Biodegradable implants and/or carriers can also be employed for delivering the compositions of the presently disclosed subject matter to various locations within a subject, including within the eye of a subject (see e.g., U.S. Pat. Application Serial Nos. 2011/0076318, 2014/0341968, 2019/0192341, and 2020/0069847).

### II.B.3. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly. After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### III. Methods and Uses

In some embodiments, the presently disclosed subject matter relates to methods for treating and/or preventing development of macular degeneration in a subject in need thereof. In some embodiments, the methods comprise, consist essentially of, or consist of administering to the subject one or more doses of fluoxetine in an amount and via a route sufficient to treat or prevent development of macular degeneration in the subject. In some embodiments, the macular degeneration is dry macular degeneration. Thus, in some embodiments the methods of the presently disclosed subject matter can be used to prevent and/or treat macular degeneration, including but not limtied to AMD, optionally dry AMD. In some embodiments, macular degeneration is characterized by damage to or breakdown of the macula, which in some embodiments, is a small area at the back of the eye. In some embodiments, macular degeneration causes a progressive loss of central sight, but not complete blindness. In some embodiments, macular degeneration is of the dry type, while in some embodiments, it is of the wet type. In some embodiments, the dry type is characterized by the thinning and loss of function of the macula tissue. In some embodiments, the wet type is characterized by the growth of abnormal blood vessels behind the macula. In some embodiments, the abnormal blood vessels hemorrhage or leak, resulting in the formation of scar tissue if untreated. In some embodiments, the dry type of macular degeneration can turn into the wet type. In some embodiments, macular degeneration is age-related, which in some embodiments is caused by an ingrowth of choroidal capillaries through defects in Bruch’s membrane with proliferation of fibrovascular tissue beneath the retinal pigment epithelium.

Treatment and/or prevention of AMD using the compositions and methods of the presently disclosed subject matter can be coupled with known methods. For example, the early and intermediate stages of AMD usually start without symptoms. A comprehensive dilated eye exam can detect AMD. The eye exam can include one or more of the following:

1. Visual acuity test. An eye chart measure is used to measure vision at distances.

2. Dilated eye exam. The eye care professional places drops in the eyes to widen or dilate the pupils. This provides a better view of the back of the eye. Using a special magnifying lens, he or she then looks at your retina and optic nerve for signs of AMD and other eye problems.

3. Amsler grid. The eye care professional also may ask you to look at an Amsler grid. Changes in central vision may cause the lines in the grid to disappear or appear wavy, a sign of AMD.

4. Fluorescein angiogram. In this test, which is performed by an ophthalmologist, a fluorescent dye is injected into the subject’s arm. Pictures are taken as the dye passes through the blood vessels in the eye. This makes it possible to see leaking blood vessels, which occur in a severe, rapidly progressive type of AMD.

5. Optical coherence tomography. This technique uses light waves, and can achieve very high-resolution images of any tissues that can be penetrated by light such as the eyes.

There are also multiple methods available for predicting susceptibility to age-related macular degeneration or geographic atrophy. As mentioned above, for example, “age-related macular degeneration or geographic atrophy” is not meant to infer that geographic atrophy is not a form or stage of age-related macular degeneration, but that a treatment or diagnosis can be in reference to the two.

Methods and biomarkers are available for predicting whether a subject is susceptible to AMD, including, for example, the existence genetic variants of complement factor H (CFH) and high-temperature requirement factor A-1 (HTRAl) that can be detected, smoking, and, of course, age. When a subject has been tested and is diagnosed or predicted to be susceptible to an RPE disease or disorder, one or more of the therapeutic agents of the presently disclosed subject matter can be administered prophylactically.

In some embodiments, the presently disclosed subject matter relates to methods for preventing and/or treating RPE degeneration in subjects in need thereof, the method comprising, consisting essentially of, or consisting of introducing into the eye, retina, and/or RPE of the subject an effective amount of a composition comprising fluoxetine, optionally wherein the composition comprises fluoxetine in a substantially enantiomerically pure form. In some embodiments, the RPE degeneration is associated with age-related macular degeneration (AMD), optionally dry AMD.

Any method of administration can be employed in order to deliver the compositions of the presently disclosed subject matter to their desired target cell(s), tissue(s), and/or organ(s), which routes of administration include but are not limited to intravitreous injection; subretinal injection; episcleral injection; sub-Tenon’s injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; and any combination thereof.

In some embodiments, the presently disclosed subject matter also relates to methods and uses of the compositions disclosed herein for treating and/or preventing development of diseases, disorders, and or conditions that are associated with NLRP3-ASC inflammasome activity in subjects. In some embodiments, the presently disclosed subject matter methods and uses comprise, consist essentially of, or consist of administering to a subject in need thereof an effective amount of fluoxetine, optionally wherein the fluoxetine is present in an enantiomerically pure form. In some embodiments, the presently disclosed subject matter relates to administering a composition to a site associated with NLRP3-ASC inflammasome activation. As used herein, the phrases “associated with NLRP3-ASC inflammasome activation” and “NLRP3 immunosome-related disorder, disease, and/or condition” refer to any disease, disorder, and/or condition at least one symptom of consequence of which results either directly or indirectly from NLRP3-ASC inflammasome activation and for which inhibition of NLRP3-ASC inflammasome activation would be expected to ameliorate or eliminate the at least one symptom of consequence.

Various diseases are known to be associated with NLRP3-ASC inflammasome activation, and include but are not limited to arthritis, optionally rheumatoid arthritis and/or reactive arthritis, diabetes mellitus, chronic obstructive pulmonary disease, inflammatory bowel disease, irritable bowel syndrome, Duchenne muscular dystrophy, graft-versus-host disease, chronic pain, proliferative vitreoretinopathy, glaucoma, multiple sclerosis, bipolar disorder, major depressive disorder, renal fibrosis, nephritis, pulmonary fibrosis, Huntington’s disease, osteoporosis, chronic lymphocytic leukemia, anxiety disorders, pulmonary tuberculosis, osteoporosis, optionally osteoporosis in postmenopausal women and/or fracture patients, systemic lupus erythematosus, discoid lupus erythematosus, chronic inflammatory and neuropathic pain, autosomal dominant polycystic kidney disease, spinal cord injury, Alzheimer’s disease, neuropathic pain, hypertension, varicose veins, type I diabetes, type II diabetes, gout, autoimmune hepatitis, graft vascular injury, atherosclerosis, thrombosis, metabolic syndrome, salivary gland inflammation, traumatic brain injury, ischemic heart disease, ischemic stroke, Parkinson’s disease, melanoma, neuroblastoma, prostate, breast, skin, and thyroid cancers, tubular early gastric cancer, neuroendocrine cancer, mucoid colon cancer, colon cancer; high-grade urothelial carcinoma, kidney clear cell carcinoma, undifferentiated ovary carcinoma, papillary intracystic breast carcinoma, gram negative sepsis, infectious Pseudomonas aeruginosa, Vibrio cholera, Legionella spp., Francisella spp., and Leishmania spp. Chlamydia spp., cryopyrinopathies; keratitis, acne vulgaris, Crohn’s disease, ulcerative colitis, insulin resistance, obesity, hemolytic-uremic syndrome, polyoma virus infection, immune complex renal disease, acute tubular injury, lupus nephritis, familial cold autoinflammatory syndrome, Muckle-Wells syndrome and neonatal onset multisystem inflammatory disease, chronic infantile neurologic cutaneous and articular autoinflammatory diseases, renal ischemia-perfusion injury, glomerulonephritis, cryoglobulinemia, systemic vasculitides, IgA nephropathy, malaria, helminth parasites, septic shock, allergic asthma, hay fever, drug-induced lung inflammation, contact dermatitis, leprosy, Burkholderia cenocepacia infection, respiratory syncitial virus infection, psoriasis, scleroderma, cystic fibrosis, syphilis, Sjögren’s syndrome, inflammatory joint disease, non-alcoholic fatty liver disease, sterile liver inflammation, cardiac surgery (peri-/post-operative inflammation), acute and chronic organ transplant rejection, acute and chronic bone marrow transplant rejection, tumor angiogenesis, amyotrophic lateral sclerosis, and autism spectrum disorder.

Inflammasomes require the adapter protein apoptosis associated speck-like protein containing a CARD (ASC) for the activation of caspase-1. After inflammasome activation, ASC assembles into a large protein complex, which is termed a “speck”. ASC specks can be observed as they reach a size of around 1 µm and in most cells only one speck forms upon inflammasome activation.

Thus, in some embodiments NLRP3-ASC inflammasome activation can result in ASC speck formation, and as such, the compositions of the presently disclosed subject matter can in some embodiments be employed to inhibit ASC speck formation in cells. Therefore, in some embodiments the compositions of the presently disclosed subject matter that comprise, consist essentially of, or consist of fluoxetine are employed to treat and/or prevent development of a disease, disorder, or condition associated with NLRP3-ASC inflammasome activation in a subject.

Accordingly, the presently disclosed subject matter also provides uses of compositions comprising, consisting essentially of, or consisting of fluoxetine, optionally substantially enantiomerically pure formulations thereof for treating and/or preventing development of diseases, disorder, and/or conditions in subjects in need thereof, optionally diseases, disorder, and/or conditions of the eye, further optionally diseases, disorder, and/or conditions of the retina, further optionally diseases, disorder, and/or conditions of the RPE, wherein the disease or disorder of the eye is associated with RPE degeneration. In some embodiments, a composition comprising, consisting essentially of, or consisting of fluoxetine is used to treat and/or prevent development of macular degeneration in a subject in need thereof. In some embodiments, the fluoxetine is substantially enantiomerically pure R-fluoxetine or substantially enantiomerically pure S-fluoxetine. In some embodiments, the subject is a human.

In some embodiments, the methods of the presently disclosed subject matter are employed as a combination therapy with other active agents that are known to provide benefit for one of the diseases, disorders, and/or conditions for which the presently disclosed subject matter compositions and methods are appropriate. Exemplary, nonlimiting compositions and methods for treating and/or inhibiting an NLRP3 immunosome-related disorder, disease, and/or condition include those disclosed in U.S. Pat. Application Publication No. 2018/0008629 and U.S. Pat. No. 10,869,880, each of which is incorporated herein by reference in its entirety.

Additionally, it has been determined that administration of racemic fluoxetine preparations to the eye can induce cataract formation. Particularly, cataract formation was observed in up to 50% of subjects to which a racemic fluoxetine preparation was administered. It was surprisingly noted that administration of either enantiomer of fluoxetine did not induce cataract formation.

As such, in some embodiments the presently disclosed subject matter relates to methods for reducing the incidence of cataract formation in subjects receiving fluoxetine, which methods in some embodiments comprise, consist essentially of, or consist of administering the fluoxetine in a substantially pure enantiomeric form rather than as a racemic mixture. It is noted that either substantially pure enantiomeric form of fluoxetine (e.g., substantially pure R-fluoxetine or substantially pure S-fluoxetine) can be employed in the compositions, methods, and uses of the presently disclosed subject matter.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods for the Examples

Cell culture. The human retinal pigment epithelial cell line ARPE-19 (ATCC) was maintained in Dulbecco’s Modified Eagle Medium (DMEM) supplemented with 10% fetal bovine serum (FBS) and standard antibiotics. Mouse bone marrow derived macrophages (BMDMs) were cultured in Iscove’s modified Dulbecco’s media (IMDM) with 10% FBS and 20% L929 supernatants. All cells were maintained at 37° C. in a 5% CO₂ environment.

Animals. Animal experiments were approved by the University of Virginia (UVA) Institutional Animal Care and Use Committee. C57BL/6J mice (The Jackson Laboratory) were anesthetized with ketamine hydrochloride and xylazine.

### Example 1

### Fluoxetine Inhibits Inflammasome Activation

In order to test for the ability of fluoxetine to inhibit inflammasome activation, cells were transfected with (SEQ ID NO: 1) RNA or mock-transfected for 12 hour and pre-treated with Fluoxetine (10 µM) for 1 hour and again after Alu RNA (SEQ ID NO: 1) transfection. Supernatants were collected and were briefly spun down to remove floating cells. Proteins from the cell-free supernatant were precipitated by adding sodium deoxycholate (0.15% final) followed by adding TCA (7.2% final) and incubating on ice overnight. Samples were spun down at 13000 × g for 30 minutes and pellets were washed 2 times with ice-cold acetone. Precipitated proteins solubilized in 4X LDS Buffer with 2-mercaptoethanol were resolved by SDS-PAGE on NOVEX® brand Tris-Glycine Gels (Invitrogen Corporation, Carlsbad, California, United States of America) and transferred onto Immobilon-FL PVDF membranes (MilliporeSigma, Burlington, Massachusetts, United States of America). The transferred membranes were blocked in 5% nonfat dry skim milk for 1 hour at room temperature and then incubated with primary antibody at 4° C. overnight.

The immunoreactive bands were visualized using species specific secondary antibodies conjugated with IRDYE®. Blot images were captured using an ODYSSEY® imaging system. The antibodies used were as follows: mouse monoclonal anti-human caspase-1 antibody (AdipoGen Life Sciences, San Diego, California, United States of America; 1:1000) and mouse monoclonal anti-mouse caspase-1 antibody (AdipoGen; 1:1000).

FIG. 1 is a representative western blot image showing Alu RNA (SEQ ID NO: 1) induced cleavage of pro-caspase-1 (50 kDa) into active caspase-1 (20 kDa) in mouse bone marrow derived macrophages (BMDMs). FIG. 1 also shows that Fluoxetine (FLX) reduced this caspase-1 activation (n = 3 per group).

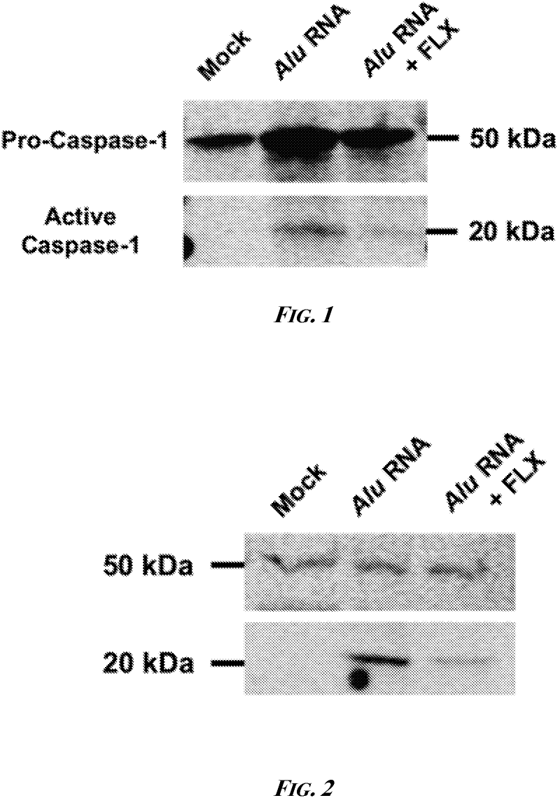

FIG. 2 is a representative western blot image showing Alu RNA (SEQ ID NO: 1) induced cleavage of pro-caspase-1 (50 kDa) into active caspase-1 (20 kDa) in human ARPE19 cells, and that Fluoxetine (FLX) reduced this caspase-1 activation (n = 3 per group).

IL-1B and IL-18 Enzyme Linked ImmunoSorbent Assays (ELISA) were also employed to assay secreted mouse IL-β and human IL-18 in the conditioned cell culture media of cells treated with B2 RNA (SEQ ID NO: 2). ELISAs employed ELISA kits (R&D Systems, Inc., Minneapolis, Minnesota, United States of America) according to the manufacturer’s instructions. Groups were compared by two-tailed Student’s t test.

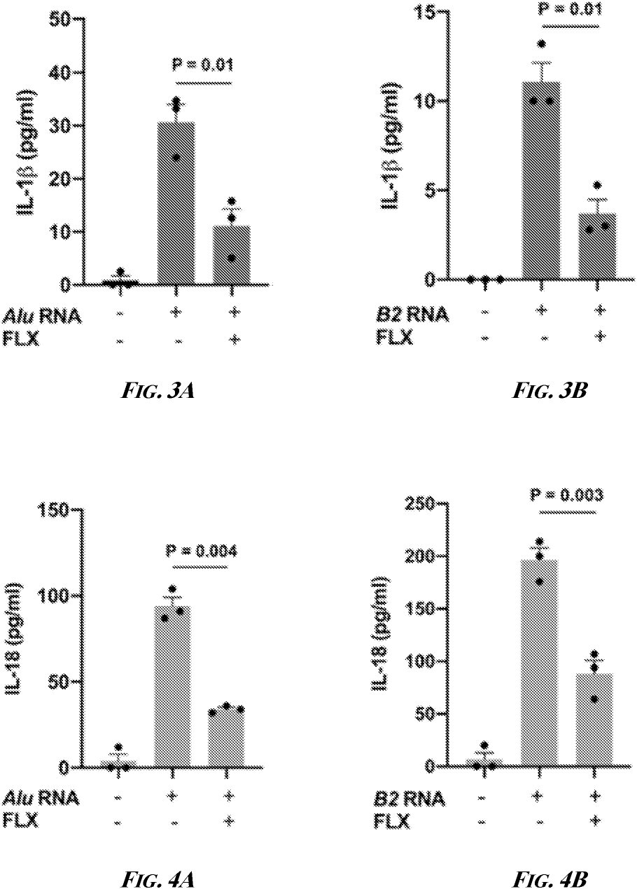

FIGS. 3A and 3B are bar graphs summarizing the results of ELISA analyses of secreted IL-18 (FIG. 3A) and secreted IL-1β (FIG. 3B) from mouse bone marrow derived macrophages (BMDMs). Alu RNA (SEQ ID NO: 1) and B2 RNA (SEQ ID NO: 2) both induced secretion of these cytokines in BMDMs, and Fluoxetine (FLX) statistically significantly reduced the secretion of both (P = 0.01 for each).

FIGS. 4A and 4B are bar graphs summarizing the results of ELISA analysis of secreted IL-18 (FIG. 4A) and secreted IL-1β (FIG. 4B) from human ARPE-19 cells. As was seen in BMDMs, Alu RNA (SEQ ID NO: 1) and B2 RNA (SEQ ID NO: 2) induced secretion of these cytokines in ARPE-1 cells, and Fluoxetine (FLX) statistically significantly reduced the secretion of both (P < 0.005 for each).

### Example 2

### Fluoxetine Inhibits ASC Speck Formation

The ASC speck assay was employed as a measure of inflammasome activation. BMDMs seeded on chambered coverslips (30,000 cells/well) for 12 hours were pretreated with Fluoxetine (10 µM) or 0.1% DMSO (control) for 2 hours. Cells were transfected with Alu RNA (SEQ ID NO: 1) using LIPOFECTAMINE™ 3000 brand transfection reagent (Thermo Fisher Scientific, Waltham, Massachusetts, United States of America) for 12 hours. Coverslips were fixed with 2% paraformaldehyde (15 minutes at room temperature), washed with PBS, permeabilized, blocked with blocking buffer (PBS, 0.1% TX-100, 5% normal goat serum; 1 hour at 4° C.) and incubated with anti-ASC antibody (Adipogen; 1:300) with blocking buffer. Nuclei were stained with DAPI. Slides mounted using FLUOROMOUNT-G® brand mounting medium (SouthernBiotech, Birmingham, Alabama, United States of America) were imaged by confocal microscopy (Nikon A1R). The numbers of specks per 40x field were compared by two-tailed Student’s t test.

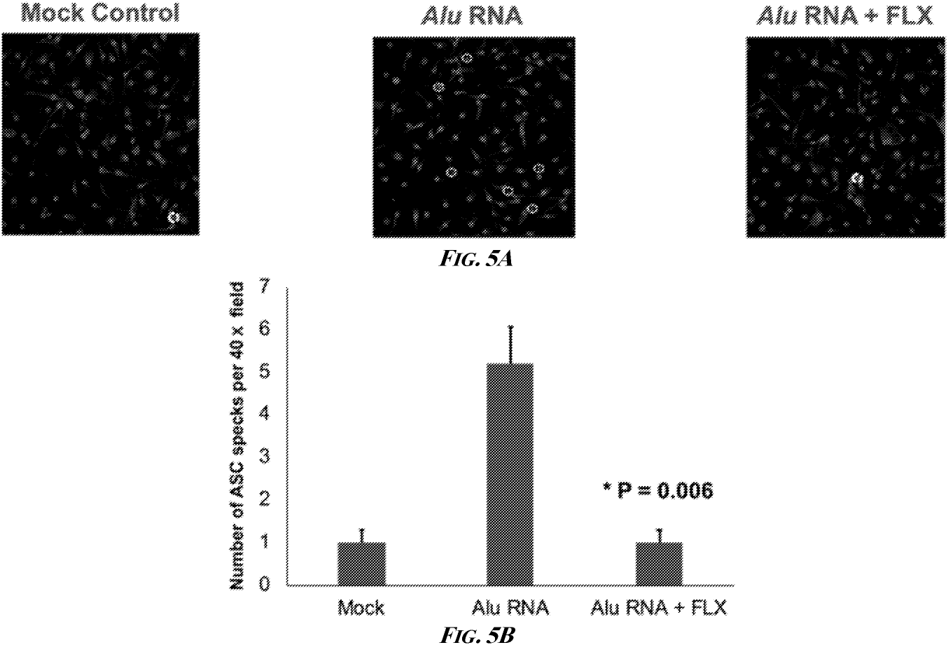

The results are presented in FIG. 5. The top panels of FIG. 5 show representative immunofluorescent images of ASC specks (stained red in color photographs and circled in white for ease of identification) in BMDMs. The bottom panel of FIG. 5 is a bar graph of mean and SEM (n = 5 per group; *P = 0.006 of Alu RNA + FLX vs. Alu RNA; Student’s two-tailed t test). As can be seen Alu RNA (SEQ ID NO: 1) induced speck formation, which was inhibited by FLX treatment.

### Example 3

### Fluoxetine Inhibits Alu RNA-Induced RPE Degeneration in Mice

The ability of various antidepressant drugs to inhibit Alu RNA (SEQ ID NO: 1) induced RPE degeneration was tested in mice. In vitro transcribed Alu RNA (SEQ ID NO: 1; 300 ng in 1 µl) or vehicle control (PBS) were injected subretinally into mice. Fluoxetine (FLX), Citalopram (CIT), Paroxetine (PAR), Sertraline (SER), Escitalopram (ESC), Venlafaxine (VEN), Imipramine (IMI), Amitriptyline (AMI), Agomelatine (AGO; all from Cayman Chemical Company, Ann Arbor, Michigan, United States of America; 1 mM in 0.5 µl), or PBS was injected into the vitreous humor 24 hours before and immediately after Alu RNA (SEQ ID NO: 1) injections. Animals were euthanized 7 days after Alu RNA (SEQ ID NO: 1) injection, and the eyes were enucleated.

RPE health was assessed by immunofluorescence staining of zonula occludens-1 (ZO-1) on RPE flat mounts. Flat mounts were fixed with 2% paraformaldehyde, stained with rabbit polyclonal antibodies against mouse ZO-1 (Invitrogen; 1:100), and visualized with Alexa-594 (Invitrogen), and imaged using an A1R Nikon confocal microscope. Images were graded as healthy or degenerated in masked fashion. Proportions of eyes with degeneration were compared using two-tailed Z test.

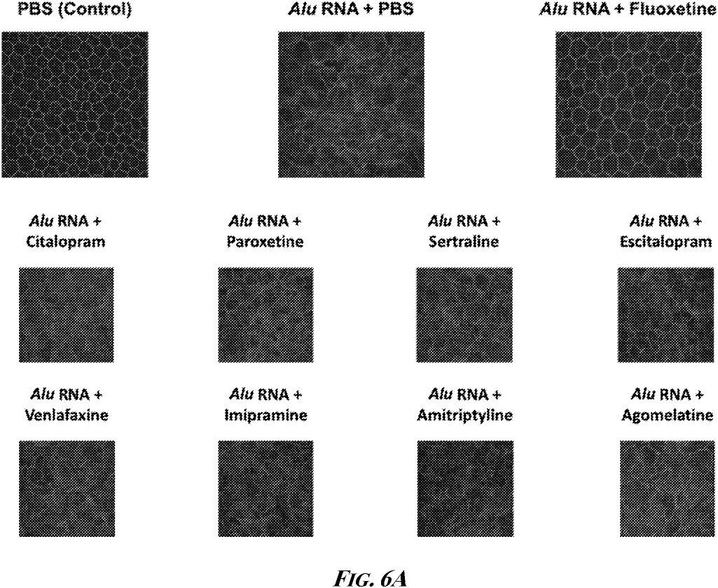

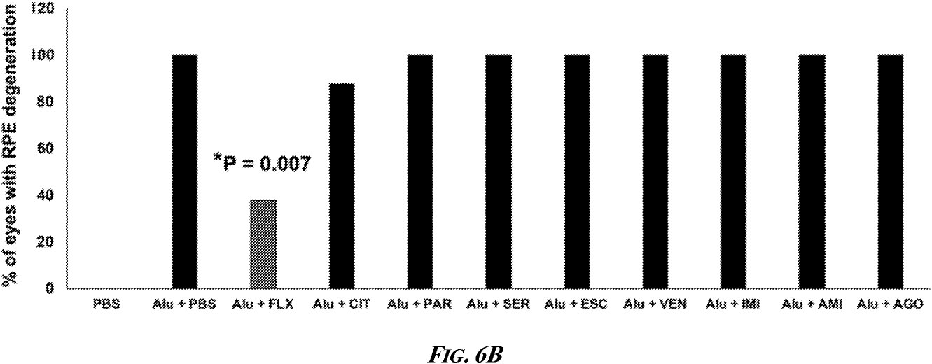

The results are shown in FIGS. 6A and 6B. In the top panel of FIG. 6A, representative ZO-1-stained (red staining in color photographs) RPE flat mounts of mice 7 days after PBS, Alu RNA (SEQ ID NO: 1) + PBS, or Alu RNA (SEQ ID NO: 1) + FLX injections. Intravitreous injection of FLX was performed 1 day before and immediately after subretinal Alu RNA (SEQ ID NO: 1) injection. The bottom two panels of FIG. 6A show similar representative ZO-1-stained (red staining in color photographs) RPE flat mounts of mice 7 days after treatment with various antidepressant drugs.

The results depicts in FIG. 6A are also summarized in FIG. 6B, which is a bar graph of the percentage of eyes with RPE degeneration in each group. 6-10 eyes were scored per group. As shown in FIG. 6B, FLX treatment significantly reduced RPE degeneration (*P = 0.007, Alu RNA + FLX vs. Alu RNA + PBS; two-tailed z-test for proportions). The other antidepressant drugs tested did not result in a statistically significant reduction in RPE degeneration.

### Example 4

### Fluoxetine Use Is Associated With Reduced Development of Dry Macular Degeneration Among People Diagnosed with Clinical Depression

Data on patients with commercial health insurance was obtained from the PearlDiver Patient Records Database (Colorado Springs, Colorado, United States of America), which captures health care claims and medication usage for persons in the Humana network from 2007 to 2017. These de-identified data are Health Insurance Portability and Accountability Act-compliant and were deemed by the University of Virginia Institutional Review Board (IRB; University of Virginia, Charlottesville, United States of America) as exempt from IRB approval requirements.

Patients were included in the analysis if they had continuous enrollment in the plan for at least 1 year and had at least 2 medical claims of Depression and were at least 50 years of age at baseline. Individuals with pre-existing Dry Macular Degeneration (1 or more medical claims prior to diagnosis of Depression) were excluded. Disease claims were identified by International Classification of Diseases (ICD)-9-CM and ICD-10-CM codes.

Individuals were classified as receiving Fluoxetine if they filled one or more outpatient pharmacy prescriptions for generic or brand versions, either in sole form or as a combination medication, as identified by National Drug Codes of the Food and Drug Administration (FDA) of the United States.

Time to initial diagnosis of Dry Macular Degeneration, which was confirmed by 2 or more medical claims, was the dependent variable for this analysis.

To analyze the risk of Dry Macular Degeneration between those exposed to Fluoxetine and those not exposed to Fluoxetine, an adjusted Cox proportional hazards model was fit. The adjusted model included as covariates age, gender, race, smoking, and body mass index. Patient data are summarized in Table 1.

TABLE 1

A Health Insurance Claims Analysis in the PearlDiver Database

| Group | | Total Patients (#) | Dry Macular Degeneration (#) | Event Rate |
|---|---|---|---|---|
| General Population | | 8,209,962 | 413,431 | 5.0% |
| Fluoxetine Users | Never | 448,075 | 21,948 | 4.9% |
| | Ever | 84,603 | 3,000 | 3.5% |

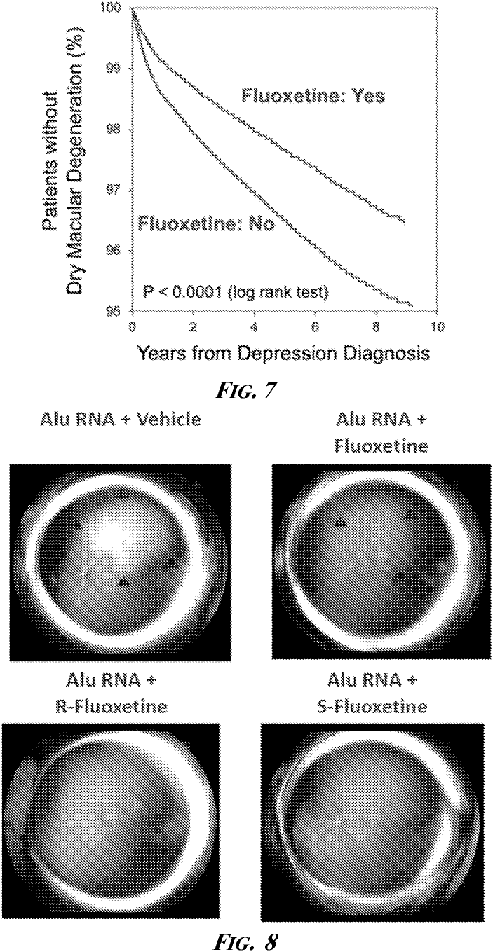

Kaplan-Meier survival plots were analyzed by log rank test for patients based on fluoxetine use, and are presented in FIG. 7 and Table 2. The event rates of dry macular degeneration in the general population and in the depression cohort are shown. Fluoxetine use was associated with a crude risk reduction of 28%. P < 0.0001 by χ2 test. Fluoxetine use was associated with a reduction in the hazard of developing dry macular degeneration. P < 0.0001 by likelihood ratio test.

TABLE 2

Multivariate Cox Proportional Hazards Regression Analysis Corrected for Age, Gender, Race, Smoking, and Body Mass Index

| Variable | Hazard Ratio (95% Confidence Interval) | P Value (Likelihood Ratio Test) |
|---|---|---|
| Fluoxetine Use (Yes vs. No.) | 0.590 (0.565, 0.616) | < 0.0001 |

Summarily, as shown in FIG. 7, fluoxetine use was associated with a slower rate of developing dry macular degeneration.

### Example 5

### Enantiomers of Fluoxetine Are More Effective in Reducing RPE Degeneration than is a Racemic Mixture of Fluoxetine

In vitro transcribed Alu RNA (SEQ ID NO: 1; 180 ng) was injected into the subretinal space of mice and 172 ng racemic fluoxetine (TCI Chemicals, Portland, Oregon, United States of America) or enantiomers of fluoxetine (Santa Cruz Biotechnology, Inc., Dallas, Texas, United States of America) was injected into the vitreous humor. Fundus photos were captured 7 days later. Representative fundus photos of the retina of mice injected with Alu RNA (SEQ ID NO: 1) and fluoxetine (racemic), R-fluoxetine enantiomer, or S-Fluoxetine enantiomer are shown in FIG. 8. Arrowheads show the extent and intensity of degeneration of the retinal pigmented epithelium (RPE). Alu RNA (SEQ ID NO: 1) induced extensive and intense RPE degeneration (white discoloration where the arrowheads are present). A racemic mixture of fluoxetine reduced the RPE degeneration extent and intensity, although the protection was partial. R-fluoxetine and S-fluoxetine individually each exerted a more complete protection against RPE degeneration.

### Example 6

### Inhibition of Amyloid Beta-Induced RPE Degeneration by Fluoxetine

The ability of fluoxetine to inhibit RPE degeneration induced by oligomerized amyloid beta was also tested. Oligomerized amyloid beta (0.6 micromoles; AnaSpec Inc., Fremont, California, United States of America) was injected into the subretinal space and fluoxetine (172 ng; TCI Chemicals) was injected into the vitreous humor. Fundus photos were captured 7 days later.

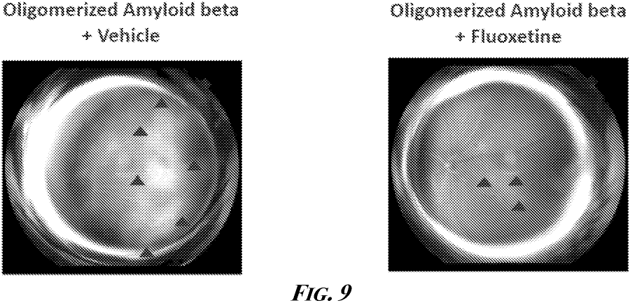

FIG. 9 is a series of fundus photos of the retina of mice injected with oligomerized amyloid beta and fluoxetine. Arrowheads show extent and intensity of degeneration of the retinal pigmented epithelium (RPE). Amyloid beta induced extensive and intense RPE degeneration (white discoloration where the arrowheads are present). Fluoxetine reduced the RPE degeneration extent and intensity.

### REFERENCES

All references listed below, as well as all references cited in the instant disclosure, including but not limited to all patents, patent applications and publications thereof, scientific journal articles, and database entries are incorporated herein by reference in their entireties to the extent that they supplement, explain, provide a background for, or teach methodology, techniques, and/or compositions employed herein. The discussion of the references is intended merely to summarize the assertions made by their authors. No admission is made that any reference (or a portion of any reference) is relevant prior art. Applicant reserves the right to challenge the accuracy and pertinence of any cited reference.

Altschul et al. (1990a) Basic local alignment search tool. J Mol Biol 215:403-410.

Altschul et al. (1990b) Protein database searches for multiple alignments. Proc Natl Acad Sci USA 87:14:5509-13.

Altschul et al. (1997) Gapped BLAST and PSI-BLAST: a new generation of protein database search programs. Nucleic Acids Res 25:3389-3402.

Devereux et al. (1984) A comprehensive set of sequence analysis programs for the VAX. Nucleic Acids Research 12:387-395.

Dong et al. (2012) Necrostatin-1 protects photoreceptors from cell death and improves functional outcome after experimental retinal detachment. Am J Pathol 181:1634-1641.

Gram (1994) Fluoxetine. N Engl J Med 331(20):1354-1361.

Kaneko et al. (2011) DICER1 deficit induces Alu RNA toxicity in age-related macular degeneration. Nature 471:325-330.

Mitchell et al. (2018) Age-related macular degeneration. Lancet 392:1147-1159.

Murakami et al. (2011) RIP kinase-mediated necrosis as an alternative mechanisms of photoreceptor death. Oncotarget 2:497-509.

Remington et al. (1975) Remington’s Pharmaceutical Sciences, 15th ed., Mack Pub. Co., Easton, Pennsylvania, United States of America.

Sato et al. (2013) Receptor interacting protein kinase-mediated necrosis contributes to cone and rod photoreceptor degeneration in the retina lacking interphotoreceptor retinoid-binding protein. J Neurosci 33:17458-17468.

Tarallo et al. (2012) DICER1 loss and Alu RNA induce age-related macular degeneration via the NLRP3 inflammasome and MyD88. Cell 149:847-859.

Trichonas et al. (2010) Proc Natl Acad Sci USA 107:21695-21700.

U.S. Pat. Application Serial Nos. 2006/0141049, 2011/0076318, 2014/0341968, 2018/0008629, 2019/0192341, 2020/0069847.

U.S. Pat. Nos. 4,018,895; 4,194,009; 7,678,078; 7,745,389; 8,642,067; 8,664,176; 9,144,516; 9,314,453; 10,004,788; 10,117,931; 10,501,473; 10,869,880.

Wong et al. (2014) Global prevalence of age-related macular degeneration and disease burden projection for 2020 and 2040: a systematic review and meta-analysis. Lancet Global Health 2:e106-116.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for treating and/or preventing development of macular degeneration in a subject in need thereof, the method comprising administering to the subject one or more doses of fluoxetine in an amount and via a route sufficient to treat or prevent development of macular degeneration in the subject.

2. The method of claim 1, wherein the macular degeneration is dry macular degeneration.

3. A method for inhibiting activation of an NLRP3-ASC inflammasome in a subject, the method comprising administering to the subject one or more doses of fluoxetine in an amount and via a route sufficient to inhibit activation of an NLRP3-ASC inflammasome in the subject.

4. The method of claim 2, wherein the NLRP3-ASC inflammasome is present in retinal pigmented epithelium (RPE) cells and/or macrophages in the subject.

5. A method for inhibiting ASC speck formation in a subject, the method comprising administering to the subject one or more doses of fluoxetine in an amount and via a route sufficient to inhibit ASC speck formation in the subject.

6. A method for inhibiting Alu RNA-induced RPE degeneration in a subject, the method comprising administering to the subject one or more doses of fluoxetine in an amount and via a route sufficient to inhibit Alu RNA-induced RPE degeneration in the subject.

7. A method for treating and/or preventing development of a disease, disorder, and/or condition associated with NLRP3-ASC inflammasome activation in a subject in need thereof, the method comprising administering to the subject one or more doses of fluoxetine in an amount and via a route sufficient to treat or prevent development of the disease, disorder, and/or condition in the subject.

8. The method of claim 7, wherein the disease, disorder, and/or condition associated with NLRP3-ASC inflammasome activation is selected from the group consisting of arthritis, optionally rheumatoid arthritis and/or reactive arthritis, diabetes mellitus, chronic obstructive pulmonary disease, inflammatory bowel disease, irritable bowel syndrome, Duchenne muscular dystrophy, graft-versus-host disease, chronic pain, proliferative vitreoretinopathy, glaucoma, multiple sclerosis, bipolar disorder, major depressive disorder, renal fibrosis, nephritis, pulmonary fibrosis, Huntington’s disease, osteoporosis, chronic lymphocytic leukemia, anxiety disorders, pulmonary tuberculosis, osteoporosis, optionally osteoporosis in post-menopausal women and/or fracture patients, systemic lupus erythematosus, discoid lupus erythematosus, chronic inflammatory and neuropathic pain, autosomal dominant polycystic kidney disease, spinal cord injury, Alzheimer’s disease, neuropathic pain, hypertension, varicose veins, type I diabetes, type II diabetes, gout, autoimmune hepatitis, graft vascular injury, atherosclerosis, thrombosis, metabolic syndrome, salivary gland inflammation, traumatic brain injury, ischemic heart disease, ischemic stroke, Parkinson’s disease, melanoma, neuroblastoma, prostate, breast, skin, and thyroid cancers, tubular early gastric cancer, neuroendocrine cancer, mucoid colon cancer, colon cancer; high-grade urothelial carcinoma, kidney clear cell carcinoma, undifferentiated ovary carcinoma, papillary intracystic breast carcinoma, gram negative sepsis, infectious Pseudomonas aeruginosa, Vibrio cholera, Legionella spp., Francisella spp., and Leishmania spp. Chlamydia spp., cryopyrinopathies; keratitis, acne vulgaris, Crohn’s disease, ulcerative colitis, insulin resistance, obesity, hemolytic-uremic syndrome, polyoma virus infection, immune complex renal disease, acute tubular injury, lupus nephritis, familial cold autoinflammatory syndrome, Muckle-Wells syndrome and neonatal onset multisystem inflammatory disease, chronic infantile neurologic cutaneous and articular autoinflammatory diseases, renal ischemia-perfusion injury, glomerulonephritis, cryoglobulinemia, systemic vasculitides, IgA nephropathy, malaria, helminth parasites, septic shock, allergic asthma, hay fever, drug-induced lung inflammation, contact dermatitis, leprosy, Burkholderia cenocepacia infection, respiratory syncitial virus infection, psoriasis, scleroderma, cystic fibrosis, syphilis, Sjögren’s syndrome, inflammatory joint disease, non-alcoholic fatty liver disease, sterile liver inflammation, cardiac surgery (peri-/postoperative inflammation), acute and chronic organ transplant rejection, acute and chronic bone marrow transplant rejection, tumor angiogenesis, amyotrophic lateral sclerosis, and autism spectrum disorder.

9. A method for preventing and/or treating RPE degeneration in a subject in need thereof, the method comprising introducing into the eye, retina, and/or RPE of the subject an effective amount of a composition comprising fluoxetine, optionally wherein the composition comprises fluoxetine in a substantially enantiomerically pure form.

10. The method of claim 10, wherein the RPE degeneration is associated with age-related macular degeneration (AMD), optionally dry AMD.

11. The method of any one of claims 1-10, wherein the fluoxetine is substantially enantiomerically pure R-fluoxetine or substantially enantiomerically pure S-fluoxetine.

12. Use of a composition comprising fluoxetine to treat or prevent development of a disease or disorder in a subject in need thereof, optionally of the eye, further optionally the retina, further optionally the RPE, wherein the disease or disorder of the eye is associated with RPE degeneration.

13. Use of a composition comprising fluoxetine to treat or prevent development of macular degeneration in a subject in need thereof.

14. The use of claim 12 or claim 13, wherein the fluoxetine is substantially enantiomerically pure R-fluoxetine or substantially enantiomerically pure S-fluoxetine.

15. The use of any one of claims 12-14, wherein the subject is a human.

16. Use of a composition comprising fluoxetine to treat or prevent development of a disease or disorder in a subject in need thereof, wherein the disease or disorder is associated with NLRP3-ASC inflammasome activation.

17. A pharmaceutical composition for preventing and/or treating a disease or disorder associated with RPE degeneration and/or NLRP3-ASC inflammasome activation in a subject in need thereof, the pharmaceutical composition comprising an effective amount of fluoxetine, optionally wherein the fluoxetine is substantially enantiomerically pure R-fluoxetine or substantially enantiomerically pure S-fluoxetine.

18. The pharmaceutical composition of claim 17, wherein the disease or disorder associated with RPE degeneration and/or NLRP3-ASC inflammasome activation is selected from the group consisting of DICER1 syndrome, type 2 diabetes mellitus, diabetic retinopathy, age-related macular degeneration (AMD), aberrant choroidal and retinal neovascularization (CRNV), subretinal and retinal fibrosis, Fuchs’ endothelial corneal dystrophy, Alzheimer’s disease, rheumatoid arthritis, lupus, renal injury, tubulointerstitial fibrosis, glial axonal degeneration, idiopathic pulmonary fibrosis, lipid dysregulation, cholesterol accumulation associated with non-alcoholic steatohepatitis, clear cell renal cell carcinoma, atopic dermatitis, glomerulopathy, disorders of hypomyelination, tubal ectopic pregnancy and tubal abnormalities such as but not limited to cysts and disorganization of epithelial cells and smooth muscle cells, amyotrophic lateral sclerosis (ALS), Duchenne’s muscular dystrophy, Sertoli cell deficiency/impaired spermatogenesis, and combinations thereof.

19. The pharmaceutical composition of claim 17 or 18, wherein the disease or disorder is age-related macular degeneration (AMD).

20. The pharmaceutical composition of any one of claims 17-19, wherein the pharmaceutical composition is formulated for administration by intravitreous injection; subretinal injection; episcleral injection; sub-Tenon’s injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; and any combination thereof.

21. A method for reducing the incidence of cataract formation in subject receiving fluoxetine, the method comprising administering the fluoxetine in a substantially pure enantiomeric form rather than as a racemic mixture.


SEQUENCE LISTING (2 sequences)

SEQ ID NO: 1 - RNA, 315 nt, Artificial Sequence
  Artificially synthesized Alu RNA
gauccuaauacgacucacuauagggccgggcgcgguggcucacgccuguaaucccagcacuuugggaggccgaggcgggcggaucacaaggucaggagaucgagaccauccuggcuaacacagugaaaccccgucucuacuaaaaauacaaaaaauuagccgggaguggugucgggcgccuguagucccagcuacucgugaggcugaggcaggagaauggcgugaacccgggagguggagcuugcagcgagccgagaucgcgccacugcacuccagccugggugacagagcgagacucugucucuuuaaagguac

SEQ ID NO: 2 - RNA, 209 nt, Artificial Sequence
  Artificially synthesized B2 RNA
gggcuggagagauggcucagugguuaagagcaccugacugcucuuccagagguccugaguucaauucccagcaaccacaugguggcucacaaccaucuguaaugagaucugaugcccucuucuggugugucugaagacagcuacaguguacuuacauauaauaaauaaauaaauaaauaaaucuuaaaaaaaaaaaaaagaaagaaaaa